(19) United States
(12) Patent Application Publication
(10) Pub. No.: US 2014/0079804 A1
(43) Pub. Date: Mar. 20, 2014

(54) COMPOSITIONS AND METHODS FOR TREATMENT OF INFECTIONS

(71) Applicant: Thompson Cooper Laboratories, LLC, Horsham, PA (US)

(72) Inventors: Eugene R. Cooper, Berwyn, PA (US); Evan R. Thompson, Horsham, PA (US); Elliot R. Thompson, Horsham, PA (US)

(73) Assignee: Thompson Cooper Laboratories, LLC, Horsham, PA (US)

(21) Appl. No.: 14/026,342

(22) Filed: Sep. 13, 2013

Related U.S. Application Data
(63) Continuation of application No. PCT/US2012/029300, filed on Mar. 15, 2012.

(51) Int. Cl. A61K 33/40; A61K 31/17; A61K 47/10; A61K 9/00

(52) U.S. Cl. CPC A61K 33/40; A61K 9/0014; A61K 31/17; A61K 47/10
USPC 424/616; 514/588

(57) ABSTRACT

This invention relates to the field of topical treatments for infections such as nail infections, particularly fungal infections of the nail commonly referred to as onychomycosis. Particularly, the invention relates to novel compositions containing peroxide for the treatment of such nail infections and other infections.

### RELATED APPLICATIONS

This application is a continuation of International Application No. PCT/US2012/029300, which designated the United States and was filed on Mar. 15, 2012, published in English, which claims the benefit of U.S. Provisional Application No. 61/453,118, filed on Mar. 15, 2011. The entire teachings of the above applications are incorporated herein by reference.

### FIELD OF THE INVENTION

This invention relates to the field of topical treatments for infections such as nail infections, particularly fungal infections of the nail commonly referred to as onychomycosis. Particularly, the invention relates to novel compositions containing peroxide for the treatment of such nail infections and other infections.

### BACKGROUND OF THE INVENTION

Onychomycosis occurs when fungi infect fingernails or toenails. A nail fungal infection may begin as a white or yellow spot under the tip of the nail, and as the fungus spreads deeper into the nail, several symptoms may develop. Such symptoms include thickened and discolored or dull nails, distorted in shape, and which may be brittle and/or ragged. Severely fungus-infected nails sometimes separate from the nail bed, a condition called onycholysis.

Fingernails and toenails may be infected by molds, fungi, and yeast. In onychomycosis, some of the infecting organisms include C. albicans, C. parappsilosis and S. brevicaulis. Other etiologic agents of oncychomycosis include Aspergillus flavus, A. candidus, A. fumigatus, A. sydowi, A. terreus, and other aspergillus species, Cephalosporum species and Fusrium oxysporum. Onychomycosis is associated with chronic paronychia where active invasion of the nail plate is less frequent. Tinea ungium is an invasive disease of the nail plate caused by a dermatophyte, most commonly T. interdigitale, T. rubrum and T. metagrophytes. Generally, tinea ungium is classified into two subtypes—leukonychia mycotica (superficial white onychomycosis, SWO) and invasive sublingual onychomycosis (commonly called ringworm of the nail). In leukonychia mycotica, the nail is invaded from the top exhibiting pitting or infected patches on the surface of the nail. This type of infection is caused by T. mentagrophytes.

Nail fungus is notoriously difficult to treat, and infection often recurs after termination of treatment because the conditions favorable to fungal growth remain (e.g., a warm, moist, dark environment, such as is frequently found with athletes' feet. Other factors also contribute to the difficulty of treatment, including the slow pace of nail growth, the hardness of the nail plate, and the site of infection being between the nail bed and plate.

The transport of molecules across membranes is in part dependent on size. The diffusion coefficient drops dramatically (exponentially) with increasing molecular weight. The nail is a relatively thick membrane and is hydrophilic. Yet current treatments for onychomycosis include lipophilic oral anti-infective agents having large molecular weights, which are therefore unsuitable for topical administration due to poor nail penetration. Such agents include terbinafine, itraconazole, and fluconazole, all of which require extended dosing periods and may be also associated with systemic toxicity, risk of liver damage, and other disadvantages.

Other treatments include topical applications, such as ciclopirox, naftifine compositions, econazole compositions, FUNGASiL® (terbinafine) and lytic peptides (see, e.g., U.S. Pat. No. 7,803,755).

Hydrogen peroxide (H₂O₂) is a well-known oxidizing anti-microbial agent. It has been recommended for use topically to treat fungal infections, including mild or non-severe nail fungal infections (see, e.g., http://www.nails-fungus.com/hydrogen-peroxide.html). Existing recommended concentrations are generally a low (3%) concentration of hydrogen peroxide to treat the skin and nail, and the treatments usually involve dipping or soaking infected toes and fingers in such a dilute solution of hydrogen peroxide.

Those of skill in the art may appreciate that higher concentrations of hydrogen peroxide may be desirable as more effective antifungal agents. However, for a variety of reasons, current recommendations do not use aqueous solutions with concentrations of H₂O₂ higher than 3% because soaking toes and fingers in such concentrated solutions is irritating to the skin. See, e.g., http://www.atsdr.cdc.gov/MHMI/mmg174.html. The art is in need of improved agents for the treatment of onychomycosis without the disadvantages of existing agents.

### SUMMARY OF THE INVENTION

The current invention allows for the localization of hydrogen peroxide on the nail of an animal or human in concentrations of about 3% or higher, and for the rapid delivery of hydrogen peroxide into the nail by using a highly volatile solvent. For example, a 3% aqueous concentration of hydrogen peroxide has no visual effect when applied as a thin film via a brush onto a normal finger nail. However, when this same amount and concentration of hydrogen peroxide is applied in a solvent system containing a highly volatile solvent, the nail shows signs of whiteness and after 15-30 minutes a slight tingling sensation may sometimes be felt.

Thus, the inventors herein have discovered that moderate concentrations of hydrogen peroxide (such as concentrations used in hair dyes) can be made to provide more effective and rapid treatment when delivered in films applied to affected nails by using highly volatile solvents, such as alcohols. Highly volatile solvents generally evaporate more than about twice as fast as water.

In another aspect of the invention, a 9% concentration of H₂O₂ in a composition containing a highly volatile solvent (such as shown in Example 1 below) showed considerably more nail whiteness as compared to the 6% concentration, showing deeper penetration and delivery.

In another aspect of the invention the composition includes peroxide, (e.g., hydrogen peroxide et al.) that exceeds 6%, a highly volatile solvent, and a thickening agent (e.g., a polymer), which is applied to the nail in the form of a liquid/gel that dries to a thin film, which for preferred polymers is easily washed off. The composition of the invention achieves penetration through the nail in minutes, rather than the days it may take other topical medications designed to treat onychomycosis. There is no need for special pretreatments of the nail or even for solvent systems to remove lacquers often used for topical nail products. The invention also includes compositions of peroxide that include hydrogen peroxide adducts/complexes, e.g., carbamide peroxide.

Thus in another aspect, the invention is a composition for treating onychomycosis having an effective amount of a peroxide, a highly volatile solvent, and a gelling/thickening agent which renders the composition suitable for applying with a brush, roller, or the like. The amount of peroxide may range from as low as about 3% to as high as about 20%. While the invention may be adapted for even higher concentrations of peroxide, such higher concentrations may pose issues with irritation. In another aspect, then, the peroxide amount may be from about 6-20%, 7-15%, 8-12%, or about 9-10%. Compositions of the invention allow a patient to determine the frequency of dosing conveniently, simply by applying until the desired effect is obtained.

Thus, in one aspect, the invention provides an anti-infective composition containing by weight at least 3% peroxide, at least 40% highly volatile solvent, and optionally water. In some aspects, the composition is an antifungal composition. In another aspect, the peroxide may be selected from the group consisting of hydrogen peroxide, peroxide complexes, and adducts thereof. In one aspect, the peroxide is hydrogen peroxide. In another aspect, the peroxide is carbamide peroxide. In yet another aspect of the invention, the peroxide is present in the composition from about 6% to 20% by weight. In another aspect, the peroxide is present from about 9% to 12%.

In some aspects, the highly volatile solvent is an alcohol. In another aspect, the alcohol may be selected from the group consisting of methanol, ethanol, n-propanol, and isopropanol. In one aspect, the alcohol is isopropanol. In some aspects, the highly volatile solvent is present from about 40% to 95%. In still other aspects, the highly volatile solvent is present from about 50% to 95%.

In some aspects, the composition of the invention also contains a thickening agent. In some aspects, the thickening agent may be selected from the group consisting of hydroxypropylcellulose, polyvinylpyrrolidones, polyvinylpyrrolidone/vinyl acetates, and glyceryl polyacrylates. In another aspect, the thickening agent is hydroxpropylcellulose. In one aspect, the thickening agent is present in an amount less than about 10%. In another aspect, the thickening agent is present from about 0.25% to 2.5%, and in another aspect the thickening agent is present at about 0.5%.

In one aspect the invention provides a method of treating fungal infections by applying a composition as described in previous aspects. In some aspects, such a fungal infection may be selected from the group consisting of onychomycosis and ringworm. In some aspects, the applying step is performed a plurality of times per day for a plurality of days, and the infection is at least partially ameliorated.

These and other objects are achieved through the present invention as exemplified and further described in the Detailed Description of the Invention below.

### DETAILED DESCRIPTION OF THE INVENTION

This invention provides a method of administering to an infected nail of an animal or human, an effective amount of a peroxide, such as hydrogen peroxide, which yields a thin film of such peroxide at a higher concentration than is possible with typical aqueous solution such as an aqueous solution of 3% H₂O₂. The invention is particularly effective for treatment of a fungus-infected nail. The unique composition of the invention provides for the site of administration being tightly controlled: i.e., only the nail receives the treatment. Peroxide is delivered rapidly into the nail within less than an hour, in contrast to the delivery of conventional antifungal topical treatments which often can be measured in days.

The composition of the invention is generally a peroxide, a highly volatile solvent, and optionally water. One preferred optional additional component is a thickening agent, which provides for even more tightly controlled site of application. Other optional additional elements may be included, such as fragrances, dyes, surfactants, etc.

As shown in the Examples below, a variety of embodiments demonstrate penetration into a normal or infected nail of peroxide from such compositions of the invention. In such application, within fifteen minutes a stinging sensation is felt beneath the nail, and the nail turns white. The compositions of this invention penetrate the nail so quickly that within thirty minutes the active peroxide has penetrated the nail and has begun to attack the noxious fungal infection.

With other known antifungal composition formulations, nail penetration is normally observed to occur within days or weeks.

The Peroxide Active Agent

Effective concentrations of peroxide range from 3-20%, 7-15%, 8-12%, or about 9-10%. In one embodiment, the peroxide is hydrogen peroxide at a concentration, by weight, of about 3-6%. In one embodiment, the peroxide is hydrogen peroxide at a concentration, by weight, of about 6-9%. In another embodiment, the hydrogen peroxide is present in a concentration of about 9-12%. In another embodiment, the hydrogen peroxide is present in a concentration of about 12-15%.

Other peroxides are equally suitable for use in the invention, including adducts of H₂O₂ such as carbamide peroxide. In some embodiments, the peroxide component of the invention comprises at least two different peroxides. For example, hydrogen peroxide may be combined with carbamide peroxide to provide an effective concentration of peroxide active agent in a composition of the invention. The peroxide component of the invention may be combined with the other components as a full strength highly concentrated form, or may be diluted with water prior to combination into the composition of the invention. For example, a 25% peroxide composition may be prepared by combining a 50% aqueous peroxide solution with an equal volume of highly volatile solvent. The thickening agent may be added next, together with any other desirable additional components, such as fragrances, dyes, and/or surfactants. The composition of the invention may also be prepared as a concentrate, requiring only dilution with water or solvent to bring to end-user working strength.

The Highly Volatile Solvent

Highly volatile solvents are those which generally evaporate at least about twice as fast as water.

The highly volatile solvent of the composition is generally a low molecular weight alcohol, but other highly volatile solvents may be adapted for use in the invention. Such highly volatile solvents may be selected from those capable of dissolving the peroxide and water components of the invention, and thus includes, but is not limited to, acetone, tetrahydrofuran, and acetonitrile.

Alkyl alcohols are generally preferred as the highly volatile solvents for use in the invention, preferably lower alkyl alcohols such as methanol, ethanol, and propanol, although other alcohols are amenable to such use. Isopropyl alcohol and ethanol are particularly useful in the invention because they are in common use and are generally regarded as safe.

The highly volatile solvent is generally present in the composition of the invention at an amount of between about 40% to 95%, by weight.

In some embodiments, the highly volatile solvent component of the invention comprises at least two or more different solvents. For example, in one embodiment, isopropyl alcohol may be combined with ethanol to produce an effective volatile solvent for use in the composition of the invention.

The Thickening Agent

The thickening agent of the composition of the invention promotes application of the composition by virtue of its film-forming properties, and retention of the composition on the nail. The thickening agents of the invention may be gelling agents, film-forming agents, and other thickeners. Appropriate thickening agents allow for the composition to be applied by a variety of applicators, including brushes, droppers, patch, and sponges. In one embodiment, the thickening agent is a polymer which, after a certain period of time, ranging from a few minutes to an hour, the user may simply wash off from the nail.

The thickening agents of the invention include polymers such as hydroxypropyl cellulose. In one embodiment, the thickening agent is hydroxypropyl cellulose, a gelling agent, e.g., Klucel HF, however, other gelling agents may be used so long as they can dissolve in the hydrogen peroxide/highly volatile solvent (and optionally water) composition. Other thickening agent polymers include polyvinylpyrrolidones, polyvinylpyrrolidone/vinyl acetates, and glyceryl polyacrylates. Those of skill in the art will appreciate the wide variety of thickening agents suitable for use in the invention.

The amount of gelling/thickening agent in the composition generally ranges from 0% to about 10%, or 0.1% to 5%, or 0.2 to 2%, or 0.3 to 1%, or about 0.5%. In one embodiment, the thickening agent is Klucel HF present at about 0.5% by weight.

The composition of the invention may also contain water as an additional component. Generally, the amount of water is less than about 60%, depending on the concentrations of peroxide, highly volatile solvent, and optional components.

Other Optional Ingredients

Other optional ingredients include thickening agents, fragrances, dyes, surfactants, excipients, and the like. Additionally, local anesthetics such as lidocaine may be components of the composition such that any mild discomfort associated with higher concentrations of peroxide penetration through the nail is ameliorated.

A Treatment Regimen

The composition of the invention may be applied as frequently as needed for effective treatment. For example, the composition can be applied once an hour two or three times each day over the course of a week to achieve healthy nail growth. Low concentrations of H₂O₂ such as 3% can be applied as frequently as every few minutes. Thus, instead of using a single dose of higher concentration, multiple applications of lower concentrations can be used. The composition may be applied with an applicator, whether a brush, a dropper, or the like. A thin film of liquid or thickened liquid is applied, and allowed to dry.

Initial penetration of the peroxide through the nail produces the commonly recognized “whitening” effect, demonstrating such penetration. Lower levels of hydrogen peroxide in the present invention can be used to treat infected skin or mucous membranes such as canker sores. Depending on the concentration of peroxide, penetration through a nail will also elicit sensations in certain subjects ranging from tingling to burning, which may be mildly painful at higher concentrations. Embodiments of the invention comprising an optional local anesthetic may be appropriate when using such higher concentrations. Effectiveness may be measured by a reduction of, or complete eradication of, the nail fungus over the course of treatment, and further evidenced by healthy nail growth.

Other embodiments, uses, and advantages of the present invention will be apparent to those skilled in the art from consideration of the specification and practice of the invention disclosed herein. The specification and examples should be considered exemplary only. The intended scope of the invention is only limited by the claims appended hereto.

### EXAMPLES

The present invention will be further understood by reference to the following non-limiting examples.

### Example 1

### A Composition of the Invention

On a weight basis, 9% hydrogen peroxide, 21% water, 69.5% isopropyl alcohol, and 0.5% Klucel HF (a thickening agent, a hydroxypropylcellulose polymer), were mixed in a vessel and allowed to thicken. (The source of the peroxide was a 30% peroxide solution.) The composition could easily be brushed onto a nail surface, which dried in approximately 1 minute, and the residual polymer could subsequently be removed by washing in water. Within a few minutes, a whitening effect of the nail was observed. Within 15 minutes, the subject noted mild pain under the nail.

### Example 2a

### A Composition of the Invention

On a weight basis, 6% hydrogen peroxide, 6% water, 87.5% isopropyl alcohol, and 0.5% Klucel HF, were mixed in a vessel and allowed to thicken. (The source of the peroxide was a 50% peroxide solution.) The composition could easily be brushed onto a nail surface, which dried in approximately 1 minute, and the residual polymer could subsequently be removed by washing in water. Within approximately one minute, a significant whitening effect of the nail was observed. Within 15 minutes, one subject noted pain under the nail, while another noted a tingling sensation. In one subject, the composition was applied twice daily to a fungus-infected nail for a period of 2 weeks, at which time new, healthy nail growth was observed. The treatment regimen was terminated.

### Comparative Example 2b

### A Composition Lacking a Highly Volatile Solvent

On a weight basis, 6% hydrogen peroxide, 93.5% water, and 0.5% Klucel HF, were mixed in a vessel and allowed to thicken. The resulting composition differed from that of Example 2a in that there was no isopropyl alcohol or any other highly volatile solvent, the quantity thereof being replaced with water. The composition could easily be brushed onto a nail surface, which dried in approximately 5 minutes, and the residual polymer could subsequently be removed by washing in water. Within 15 minutes of application, no whitening effect of the nail was observed, nor were any sensations of tingling, burning, or pain observed by the patient.

### Example 3

### A Composition with Insufficient Initial Concentration of Hydrogen Peroxide

On a weight basis, 3% hydrogen peroxide, 3% water, 93.5% isopropyl alcohol, and 0.5% Klucel HF, were mixed in a vessel and allowed to thicken. (The source of the peroxide was a 50% peroxide solution.) The composition could easily be brushed onto a nail surface, which dried in approximately 1 minute, and the residual polymer could subsequently be removed by washing in water. After even 15 minutes of application no significant whitening effect of the nail was observed. However, if three applications of the 3% H₂O₂ are applied about two minutes apart, significant whitening of the nail could be seen, and after 15-30 minutes a slight sensation could be felt beneath the nail.

### Example 4

### A Composition of the Invention

On a weight basis, 9% hydrogen peroxide, 9% water, 81.5% isopropyl alcohol, and 0.5% Klucel HF, were mixed in a vessel and allowed to thicken. (The source of the peroxide was a 50% peroxide solution.) The composition could easily be brushed onto a nail surface, which dried in approximately 1 minute, and the residual polymer could subsequently be removed by washing in water. Within a few minutes, a significant whitening effect of the nail was observed.

### Example 5

### A Composition of the Invention

On a weight basis, 12% hydrogen peroxide, 12% water, 75.5% isopropyl alcohol, and 0.5% Klucel HF, were mixed in a vessel and allowed to thicken. (The source of the peroxide was a 50% peroxide solution.) The composition could easily be brushed onto a nail surface, which dried in approximately 1 minute, and the residual polymer could subsequently be removed by washing in water. Within one minute, a significant whitening effect of the nail was observed. Within 15 minutes, one subject noted pain (a burning sensation) under the nail.

### Example 6

### A Composition of the Invention

On a weight basis, 18.5% carbamide peroxide, 3.0% glycerol, 73.1% ethyl alcohol, 4.5% luviskol VA 64-polyvinylpyrrolidone vinyl alcohol, and 0.9% Klucel HF, were mixed in a vessel and allowed to thicken. The source of peroxide was solid carbamide peroxide, which is only soluble in isopropyl alcohol to about 10% and in ethanol to about 20%, therefore luviskol VA 64-polyvinylpyrrolidone vinyl alcohol is added to supersaturate the carbamide peroxide, and glycerol is added to retain the carbamide peroxide in solution following ethanol evaporation. This composition did show a modest level of precipitation upon overnight rest at 60° F.

Subsequent experimentation proved that replacing the ethyl alcohol with methanol successfully retained the carbamide peroxide in solution.

The composition (with ethanol rather than methanol) could easily be brushed onto a nail surface, which dried in approximately 5 minutes, and the residual polymer could subsequently be removed by washing in water. Within one minute some whitening effect of the nail was observed. After 15 minutes, the subject reported no burning, tingling, or other painful sensation.

### Example 7

### A Composition of the Invention

On a weight basis, 6% hydrogen peroxide, 54% water, and 40% isopropyl alcohol were mixed in a vessel and allowed to thicken. (The source of the peroxide was a 50% peroxide solution.) The composition could easily be brushed onto a nail surface, which dried in approximately 1 minute. Within approximately one minute, some whitening effect of the nail was observed. The present invention is not to be limited in scope by the specific embodiments described above, which are intended as illustrations of aspects of the invention. Functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited references are hereby incorporated by reference.

1. An anti-infective composition comprising by weight at least 3% peroxide, at least 40% highly volatile solvent, and optionally water.

2. The composition of claim 1, which is an antifungal composition.

3. The composition of claim 1, wherein the peroxide is selected from the group consisting of hydrogen peroxide, peroxide complexes, and adducts thereof.

4. The composition of claim 3, wherein the peroxide is hydrogen peroxide.

5. The composition of claim 3, wherein the peroxide is carbamide peroxide.

6. The composition of claim 1, wherein the highly volatile solvent is an alcohol.

7. The composition of claim 5, wherein the alcohol is selected from the group consisting of methanol, ethanol, n-propanol, and isopropanol.

8. The composition of claim 7, wherein the alcohol is isopropanol.

9. The composition of claim 1, further comprising a thickening agent.

10. The composition of claim 9, wherein the thickening agent is selected from the group consisting of hydroxypropylcellulose, polyvinylpyrrolidones, polyvinylpyrrolidone/vinyl acetates, and glyceryl polyacrylates.

11. The composition of claim 10, wherein the thickening agent is hydroxpropylcellulose.

12. The composition of claim 1, wherein the peroxide is present from about 3% to 20%.

13. The composition of claim 12, wherein the peroxide is present from about 6% to 15%.

14. The composition of claim 13, wherein the peroxide is present from about 9% to 12%.

15. The composition of claim 1, wherein the highly volatile solvent is present from about 40% to 90%.

16. The composition of claim 1, wherein the thickening agent is present in an amount less than about 10%.

17. The composition of claim 16, wherein the thickening agent is present from about 0.25% to 2.5%.

18. A method of treating infections, comprising the step of applying the composition of claim 1.

19. The method of claim 18, wherein the infection is selected from the group consisting of fungal onychomycosis, ringworm, and canker sores.

20. The method of claim 19, wherein the applying step is performed a plurality of times per day for a plurality of days, whereby the infection is at least partially ameliorated.
